(19) United States
(12) Patent Application Publication
(10) Pub. No.: US 2005/0171070 A1
(43) Pub. Date: Aug. 4, 2005

(54) Stable salts of o-acetylsalicylic acid containing basic amino acids II

(76) Inventors: Felix Ecker, Morris Plains, NJ (US); Ralf Bullesbach, Hackettstown, NJ (US); Wolfram Ledwoch, Langenfeld (DE); Gerhard Franckowiak, Henselweg (DE); Marianne Petersen-Braun, Konigswinter (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/501,677

(22) PCT Filed: Jan. 7, 2003
(86) PCT No.: PCT/EP03/00059

(30) Foreign Application Priority Data
Jan. 18, 2002 (DE) 1021020191

(51) Int. Cl. A61K 31/60; A61K 31/198

(52) U.S. Cl. 514/165; 514/561

(57) ABSTRACT

The present invention relates to improved compositions comprising stable salts of O-acetylsalicylic acid with basic amino acids, to pharmaceuticals comprising them and to their use for preparing pharmaceuticals.

The present invention relates to improved compositions comprising stable salts of O-acetylsalicylic acid with basic amino acids, to pharmaceuticals comprising-them and to their use for preparing pharmaceuticals.

The analgesic, antipyretic and anti-inflammatory action of acetylsalicylic acid has been known for a long time. Thus, O-acetylsalicylic acid is used as an analgesic, antipyretic, anti-rheumatic, and also as a non-steroidal anti-inflammatory agent, for example for treating arthritis, neuralgia and myalgia.

Even after more than 100 years, acetylsalicylic acid is the standard therapeutic in self-medication for the therapy of pain and fever, in particular headaches, migraine and symptoms associated with a cold, such as headaches, sore throat and pain in the limbs.

However, O-acetylsalicylic acid is only soluble to a limited extent, and as a consequence, the rate of absorption is slow. In particular in the case of pain, especially in the case of migraine, for the therapeutic effect to set in, a rapid increase of the concentration of the active compound in the body is desired and required. Hitherto, this could only be achieved by suitable administration forms, such as, for example, buffered effervescent tablets or chewable tablets.

One way of rapidly achieving high blood concentrations of the active compound is to increase the rate of dissolution of the active compound itself. This can be achieved using salts of acetylsalicylic acid. Moreover, it has to be emphasized that, in the case of long-term oral administration, the O-acetylsalicylates are tolerated well.

Known salts of acetylsalicylic acid are, inter alia, salts of acetylsalicylic acid with basic amino acids. The salt of acetylsalicylic acid with the amino acid lysine is used therapeutically. The most frequently used ASA-lysinate-comprising, medicament is an administration form for parenteral administration. It is commercially available under the name Aspisol®.

By administering ASA-lysinate orally, it is possible to achieve an increase in the concentration of active compound which almost corresponds to the blood concentration curve for a bolus injection. This has been described in the literature [Ch. Raschka, H. J. Koch, Perfusion 6 (2000), volume 13, Verlag PERFUSION, Nuremberg]. The active compound dissolves extremely rapidly and is immediately absorbed by the body.

On the market, ASA-lysinate is, as oral administration form, only obtainable as a powder/as granules (for example Delgesic®, Aspegic®). It has hitherto not been possible to prepare solid oral administration forms. The stability of these formulations was too low, so that the shelf-life of these formulations was insufficient for marketing.

The low stability of the O-acetylsalicylates is to be attributed to a back reaction of the product to O-acetylsalicylic acid and the corresponding amino acid, which back reaction is known to the person skilled in the art. The amino-acid then reacts with the O-acetylsalicylic acid with removal of the acetyl group (amidolysis) and release of the salicylic acid. However, the presence of salicylic acid in pharmaceutical preparations is undesirable and therefore to be restricted to a low, acceptable value. It is known that this degradation reaction is pH-dependent [F. Moll, Arch. Pharm. 318 (1985), 120-127]. A lowering of the pH leads to an increased protonation of the amino acid released, so that this is not available or only available to a very restricted extent for the subsequent reaction with the O-acetylsalicylic acid. The amidolysis and thus the release of salicylic acid is thereby suppressed.

Moreover, problems with respect to formulation have to be taken into account. Acetylsalicylates are readily compacted; however, their flow properties are insufficient, and they have a strong tendency to adhere to the press tools of tablet presses. These problems are solved by addition of flow improvers, lubricants and release agents, usually magnesium stearate. However, from the literature it is known to the person skilled in the art that acetylsalicylic acid and magnesium stearate are incompatible (P. C. Schmidt, Wirk- und Hilfsstoffe für Rezeptur, Defektur und Groβherstellung, Wiss. Verl.-Ges., Stuttgart 1999). In some cases, it is possible to overcome the problems by filling the active compound into hard gelatin capsules. In the present case, however, this is also problematic, since it is known that acetylsalicylic acid and gelatin are incompatible.

The non-prior-published international patent application PCT/EP01/07669 describes salts of O-acetylsalicylic acid with basic amino acids, which salts have increased stability and do therefore not have the disadvantages of the prior-art O-acetylsalicylates with respect to storage and/or sterilizability. The salts are prepared by a special process and have an average particle size above a particle size of 160 μm and a proportion of more than 60% of the particles having a particle size in the range from 100 to 200 um in a particle size distribution measured using a Malvern 2600D apparatus under standard conditions. They may comprise a certain amount of added glycine and are suitable for use as pharmaceuticals and for their preparation, for example in the form of tablets, chewable tablets or capsules for oral administration.

The particle size analysis of the O-acetylsalicylates described in the international patent application PCT/EP01/07669 differ considerably and advantageously from the O-acetylsalicylates of the prior art. The distribution of the particle sizes in these O-acetylsalicylates is narrower, and the average particle size has been shifted to larger particle dimensions. This means that these O-acetylsalicylates are comprised of larger crystals of a more uniform form (grown crystals). In addition to a narrower particle size distribution and a larger average particle size, these O-acetylsalicylates have a well-defined crystal structure. In comparison, the crystal structure of the commercially available O-acetylsalicylate Aspisol® is considerably less well defined. As a result of these advantageous properties of these O-acetylsalicylates, the residual moisture content of these O-acetylsalicylates can be kept extremely low, and it is therefore possible to suppress the above-described back reaction of the O-acetylsalicylates to O-acetylsalicylic acid and the amino acid in question. This is even more, surprising since O-acetylsalicylates have been described as being hygroscopic per se. However, these O-acetylsalicylates have reduced hygroscopicity and are, at the same temperature, considerably more stable than customary acetylsalicylates having the higher content of residual moisture.

It has now been found that it is much easier to process the O-acetylsalicylates described in the international patent application PCT/EP01/07669, while maintaining their stability advantages, to single-dose solid oral administration forms of sufficient stability by addition of a flow improver for improving flow properties and/or by subjecting them to a granulation process.

Accordingly, the invention provides a composition comprising a salt of O-acetylsalicylic acid with a basic amino acid, which salt has an average particle size above a particle size of 160 μm and a proportion of more than 60% of the particles having a particle size in a range from 100 to 200 μm in a particle size distribution measured using a Malvern 2600D apparatus under standard conditions, characterized in that the composition additionally comprises a flow improver and/or is granulated.

The invention furthermore provides a pharmaceutical comprising at least one composition according to the invention.

Finally, the invention also provides the use of compositions according to the invention for preparing pharmaceuticals for certain applications.

The present invention is illustrated in greater detail by the accompanying figures, in which:

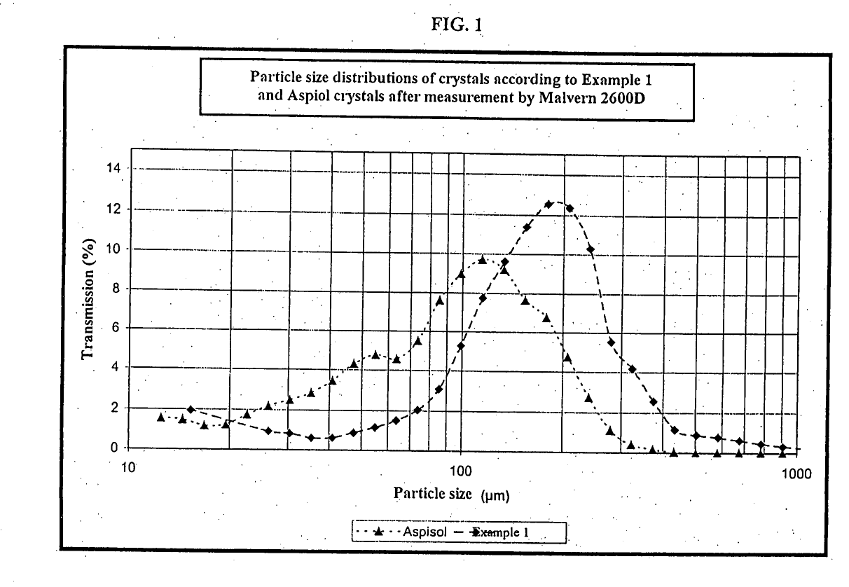

FIG. 1 shows a graphic representation of the particle size distribution of the O-acetylsalicylate prepared according to Example 1 in comparison with the particle size distribution of a commercially available O-acetylsalicylate (Aspisol®).

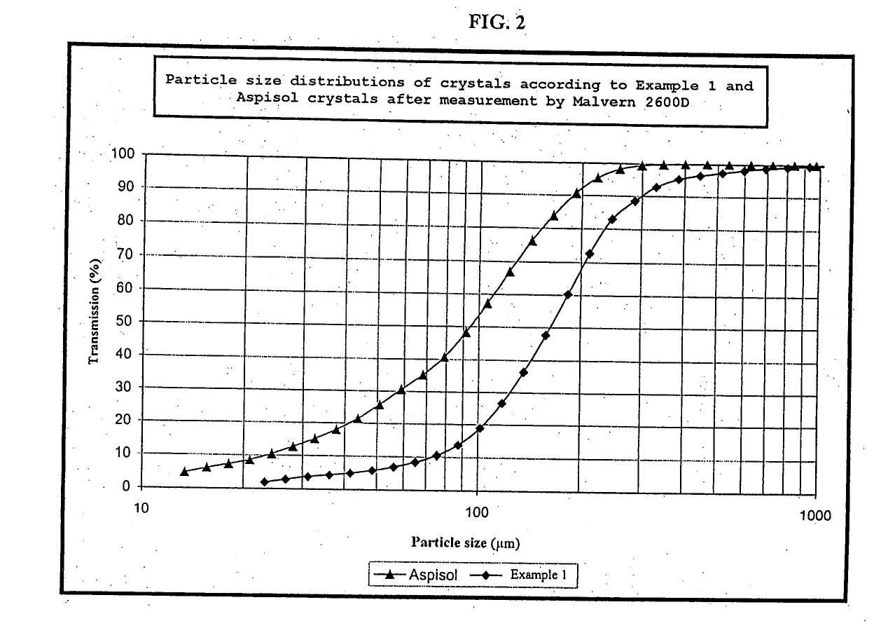

FIG. 2 shows the integrals of the curves of the particle size distributions shown in FIG. 1 for Example 1 according to the invention and Aspisol®.

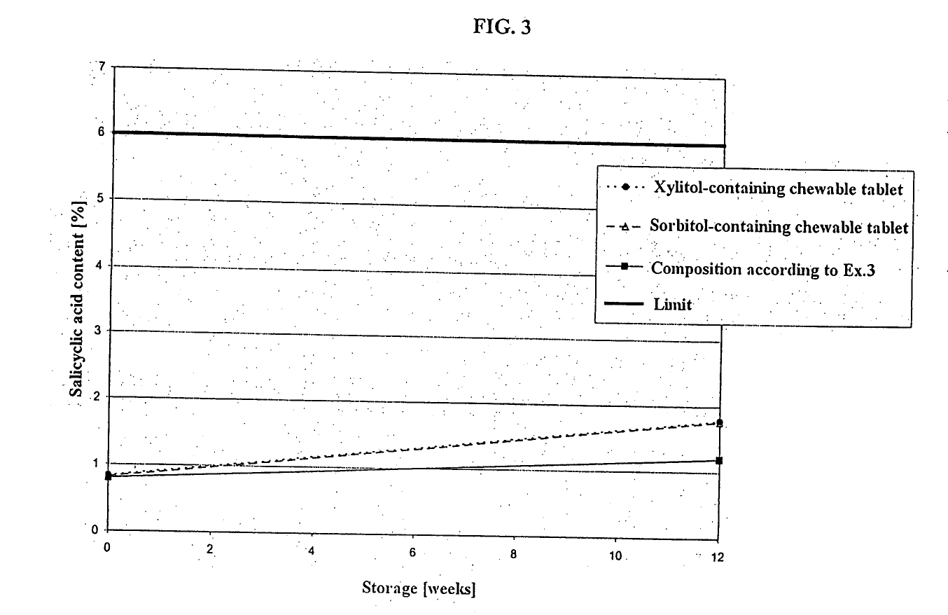

FIG. 3 shows a graphic representation of the stability of novel chewable tablets according to Example 4. What is stated is the change of the content of free salicylic acid (in %) over a storage period of 12 weeks at a temperature of 25° C. and a relative atmospheric humidity of 60%.

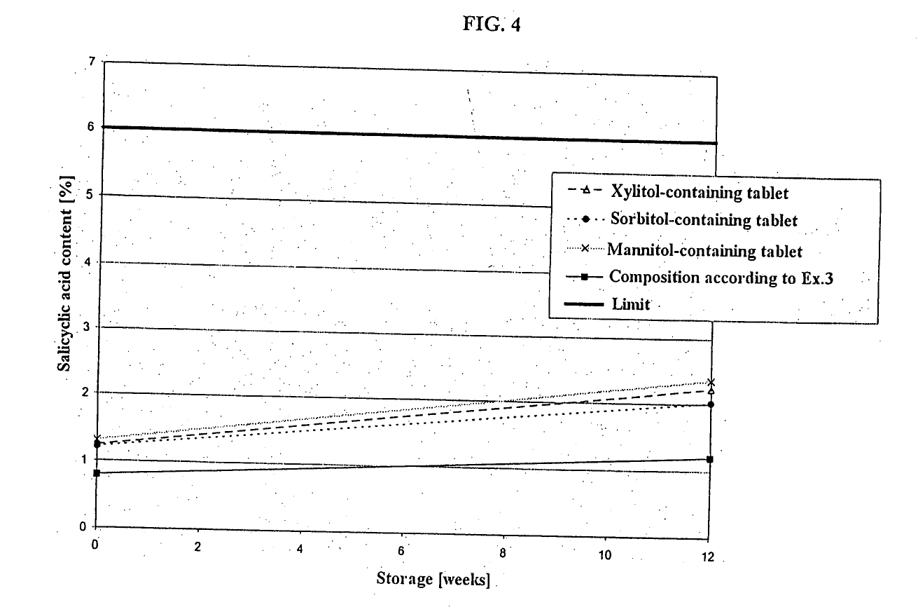

FIG. 4 shows a graphic representation of the stability of novel tablets according to Example 5. What is stated is the change of the content of free salicylic acid (in %) over a storage period of 12 weeks at a temperature of 25° C. and a relative atmospheric humidity of 60%.

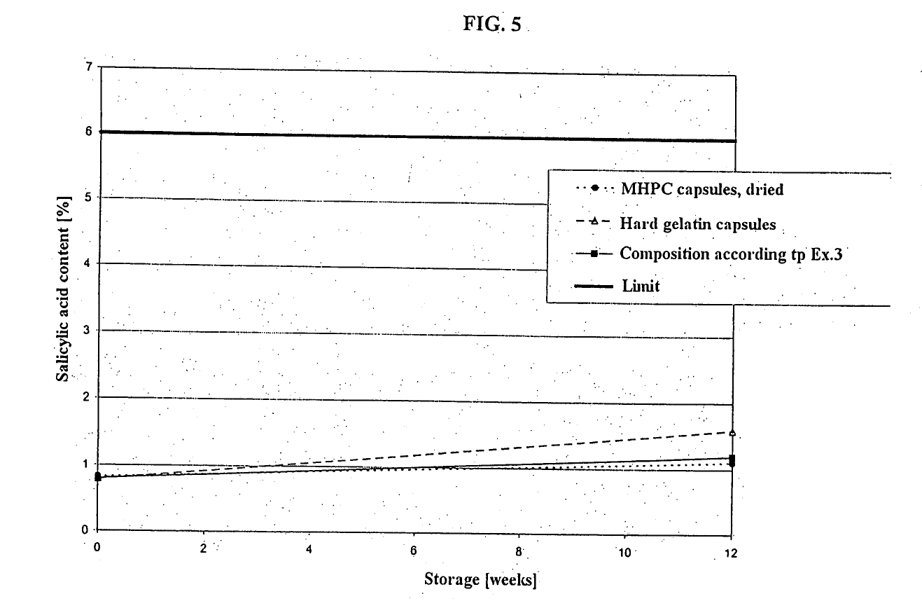

FIG. 5 shows a graphic representation of the stability of novel capsules according to Example 6. What is stated is the change of the content of free salicylic acid (in %) over a storage period of 12 weeks at a temperature of 25° C. and a relative atmospheric humidity of 60%.

Preference according to the invention is given to compositions in which the salt of O-acetylsalicylic acid with a basic amino acid comprised therein has an average particle size above a particle size of 170 μm and a proportion of more than 70% of the particles having a particle size in a range from 100 to 200 μm in a particle size distribution measured using a Malvern 2600D apparatus under standard conditions. The salt usually has a residual moisture content of less than 0.4%, preferably of less than 0.3% and in particular of less than 0.15%, of water. The low residual moisture content results in an improved stability of the compositions and pharmaceuticals according to the invention. It is possible to add a certain amount of glycine to the O-acetylsalicylate, as specified in more detail below with respect to the preparation process.

The basic amino acids suitable according to the invention as a component of the salt of O-acetylsalicylic acid can occur in the L or in the D configuration or else as a mixture of the D and the L form. The term “amino acids” designates, according to the invention, in particular the α-amino acids occurring in nature, but moreover also includes their homologues, isomers and derivatives. Enantiomers can be mentioned as an example of isomers. Derivatives can be, for example, amino acids provided with protective groups. Typical examples of basic amino acids which may be mentioned are: lysine, arginine, ornithine, diaminobutyric acid. The salt of acetylsalicylic acid with lysine is particularly suitable.

According to the invention, flow improvers are to be understood as meaning the auxiliaries which are frequently also referred to as flow agents and which are added to pulverulent or granulated, in particular hygroscopic, substances to prevent them from lumping or sticking together, thus ensuring lasting free flow (fluidification).

Preferred flow improvers are finely divided silica, microcrystalline cellulose and saccharides; and mixtures thereof. Particularly preferred flow improvers are the saccharides mannitol, sorbitol, xylitol and lactose and mixtures thereof. The flow improvers are usually employed in an amount, based on the amount of O-acetylsalicylate, of from 1 to 70% by weight, preferably from 1 to 50% by weight. However, if required, the amount of flow improver may also be higher. Thus, in a particular embodiment of the invention, in a pharmaceutical prepared from a composition according to the invention, for example in the form of a chewable tablet, the content of flow improver, in particular of saccharides, based on the pharmaceutical can be up to 70% by weight and/or 1 to 2 or even 2.5 times the amount by weight of O-acetylsalicylate.

Processes suitable for granulation are, according to the invention, the known customary processes. Preferred granulation processes are wet- and dry granulation, in particular roller compacting. Accordingly, in a preferred embodiment, the composition according to the invention is dry-granulated and in particular roller-compacted. To improve the intermediate, it is also possible to use auxiliaries for the granulation (for example binders, solvents, saccharides, polysaccharides).

In the simplest case, the pharmaceutical according to the invention is a composition according to the invention comprising the O-acetylsalicylate and a flow improver or comprising the granulated O-acetylsalicylate.

The pharmaceutical according to the invention is preferably provided as a single-dose solid oral administration form, in particular as a tablet, a chewable tablet, a soluble tablet, an enteric-coated tablet, a capsule or a colon-targeted formulation.

Both the customary two-piece hard gelatin capsules and two-piece capsules made of HPMC (hydroxypropylmethylcellulose) are suitable for the capsule version. The two-piece HPMC capsules can be used directly or after drying. Surprisingly, the O-acetylsalicylates are stable not only in the two-piece HPMC capsules but also in the two-piece hard gelatin capsules. This is another proof of the stability of the compositions and pharmaceuticals according to the invention. Furthermore, two-piece capsules made of other polymers (for example starch, cellulose, hydroxypropylcellulose, hydroxypropylcellulose lactate, hydroxypropylcellulose glycolide and hydroxyethylhydroxypropylcellulose) are also suitable.

In addition to the O-acetylsalicylate, the pharmaceutical according to the invention may, additionally to the flow improver and glycine, comprise further auxiliaries and/or active compounds. In the manufacture of the pharmaceutical, the further auxiliaries and/or active compounds may be added from a composition according to the invention; however, some or all of them may be added even in the preparation of the composition according to the invention involving, if appropriate, their granulation, so that they are already included in the composition according to the invention.

Thus, for example, it is possible to add, as a flow improver, lubricant and release agent for tableting, magnesium stearate in an amount of usually up to 2% by weight. Surprisingly, this has not reduced the stability of the formulation. If no magnesium stearate is added, the forms in the tablet press are separated by external lubrication. To this end, minute amounts of magnesium stearate are sprayed onto the punches and the wall of the die. This strongly reduces the contact between the active compound O-acetylsalicylate and the magnesium stearate. It is also possible to use other flow improvers, lubricants and release agents, for example fumaric acid and/or adipic acid.

In a particular embodiment, the pharmaceutical according to the invention comprises, as auxiliaries, exclusively water-soluble auxiliaries, preferably the saccharides described above as flow improvers, in particular selected from the group consisting of mannitol, sorbitol, xylitol and lactose and their mixtures. By selecting exclusively water-soluble auxiliaries, it is possible to prepare, in addition to tablets and chewable tablets, also soluble tablets; These dissolve rapidly and completely in water and give a clear solution. This is also the ideal base for FDT (fast dissolve tablets) preparations. In a further embodiment of the invention, the pharmaceutical is therefore completely soluble in water.

In another particular embodiment, the pharmaceutical according to the invention is free of effervescent mixtures.

The pharmaceuticals may furthermore comprise colorants (for example inorganic pigments, such as iron oxide), and also flavour and/or odour corrigents, in particular sweeteners (for example aspartame, saccharine and/or acesulfam).

In a particular embodiment of the invention, the pharmaceuticals may, in addition to salts of O-acetylsalicylic acid with basic amino acids, also comprise one or more further pharmaceutically active compounds in effective amounts, in particular one or more ADP receptor antagonists (for example ticlopidine and clopidogrel), GPIIb/IIIa receptor antagonists (for example abciximab, eptifabitide, tirofiban, orofiban, xemilofiban and sibrafiban), phosphodiesterase inhibitors (for example dipyridamole), thrombin receptor antagonists (for example hirudin, hirulog and argatroban), factor Xa inhibitors (for example antistatin, DX-9065 and penta-saccharide), HMG-CoA receptor antagonists (for example cerivastatin, simvastatin) and/or calcium antagonists (for example nifedipine).

The pharmaceuticals according to the invention can be employed as analgesics, antipyretics, anti-rheumatics, and also as nonsteroidal anti-inflammatory pharmaceuticals, for example for the treatment of diseases of the rheumatic type, arthritis, neuralgia and myalgia and/or migraine. In particular, however, they can also be employed as platelet aggregation inhibitors in the prevention and therapy of cardiovascular and cerebrovascular diseases, for example in ischaemic heart diseases, stroke, stable and unstable angina pectoris, myocardial infarction (for example acute myocardial infarction), bypass operations, PTCA (percutaneous transluminal coronary angioplasty) and/or stent implantations. Further application areas are the stimulation of the immune system in HIV patients and tumour prophylaxis (for example carcinoma of the colon, oesophagus or lung), slowing of the cognitive deterioration associated with dementia (for example Alzheimer's disease), inhibition of gallstone formation and the treatment of diabetic diseases.

In general, it has proved advantageous both in human and in veterinary medicine to administer; the active compound(s) mentioned above in total amounts of approximately 0.5 to approximately 500, preferably 5 to 100, mg/kg of body weight every 24 hours, if appropriate in the form of several individual doses, to achieve the desired result. An individual dose of an oral pharmaceutical according to the invention comprises the active compound(s) mentioned above preferably in amounts of from approximately 1 to approximately 80, in particular 2 to 30, mg/kg of body weight.

Preparation of the O-acetylsalicylate

The O-acetylsalicylates according to the invention can be prepared by the process described below. All starting materials are commercially available.

Solutions of the reactants, i.e. of O-acetylsalicylic acid and the corresponding amino acid, are combined as quickly as possible at a temperature below 30° C., preferably from 20 to 25° C., under atmospheric, pressure, and mixed to give a homogeneous phase. Suitable solvents for the reactants are water and/or water-miscible organic solvents, such as, for example, alcohols, such as methanol, ethanol or isopropanol, in particular ethanol, ethers, such as tetrahydrofuran (THF) or ketones, such as acetone.

The amounts of reactants employed are such that a slight excess of the basic amino acid is present. Preference is given to a ratio of O-acetylsalicylic acid to amino acid of from 0.1:1.05 to 1:1.5, and a ratio of O-acetylsalicylic acid to amino acid of from 1:1.05 to 1:1.2 is particularly preferred.

The O-acetylsalicylic acid solution should have a content of from 1 to 10% by weight, preferably from 5 to 10% by weight and particularly preferably from 6 to 8% by weight, of O-acetylsalicylic acid. The solution of the basic amino acid should have a content of from 10 to 40% by weight, preferably from 15 to 35% by weight and particularly preferably from 20 to 30% by Weight, of amino acid.

The O-acetylsalicylate according to the invention is then crystallized from the resulting homogeneous solution, if appropriate with addition of seed crystals, by adding a large excess, compared to the reactants, of acetone, for example an excess of from 20 to 50%, preferably from 30 to 0.40%. It is extremely important that the temperature of the crystallization phase is kept within limits which are as narrow as possible. The temperature must not exceed 40° C. and should preferably be maintained below 35° C. Preferred according to the invention is a temperature below 25° C., in particular of 0° C. Suitable for use as seed crystals are crystals of the desired product, for example Aspisol® crystals. The crystallization is carried out under atmospheric pressure.

For the process, it is of equal importance to maintain a certain mixing energy during crystallization. The homogeneous mixture of the starting materials may only be stirred gently. The mixing energy applied should not exceed 0.1 W per litre of reaction medium. According to the invention, the mixing energy applied is preferably from 0.04 to 0.06 W per litre of reaction medium. Suitable stirrers are all conventional stirring apparatuses which can be regulated in an appropriate manner, such as, for example, a stirring unit container with flow spoilers.

For crystallization, the solution should be kept under the conditions indicated above for not longer than 20 hours. According to the invention, a crystallization time of less than 10 hours under the conditions indicated above is preferred, and a time of from 1 to 8 hours is particularly preferred.

If desired, the O-acetylsalicylate according to the invention may also comprise glycine. The amount of glycine is freely selectable. According to the invention, a proportion of from 5 to 30% by weight, particularly preferably from 5 to 15% by weight and especially preferably of 10% by weight, of glycine in the reaction solution is preferred, based on the total amount of O-acetylsalicylate and glycine.

According to the present invention, the glycine can be added to the reaction mixture of the reactants as a solution in water or a water-miscible organic solvent, where the solvents described above are suitable for use as organic solvents. With respect to these reactants, glycine is inert. Under the abovementioned conditions, it is thus possible to carry out processes of crystallization of the two solids (O-acetylsalicylate and glycine) from the homogeneous phase (cocrystallization).

However, the glycine can also be added in the form of a suspension to an already crystallized suspension of the O-acetylsalicylate. The glycine suspension can be prepared in a conventional manner. According to the invention, the preparation of a glycine suspension from a solvent mixture of water and an alcohol, such as, for example, ethanol, is preferred.

The way in which the glycine is added has no influence on the properties of the O-acetylsalicylate according to the invention. It is to be noted that the addition of glycine to the O-acetylsalicylates according to the invention is not necessary. In particular, the presence of glycine has no influence on the stability of the O-acetylsalicylates according to the invention.

The crystallisate is then isolated in a conventional manner, for example by filtering or centrifuging. The solid is washed repeatedly with organic solvents, where, according to the invention, preference is given to using alcohols, such as, for example, ethanol and/or ketones, such as acetone, or mixtures of alcohols or ketones, for example mixtures of ethanol and acetone, or using various solvents of this type.

The solid is then dried under reduced pressure. The temperature here should be kept below 50° C., preferably below 40° C. and particularly preferably below 35° C. A pressure of less than 50 mbar, preferably of less than 30 mbar, should be applied to the solid. The drying can be carried out under conventional conditions, for example in a drying apparatus.

The process according to the invention can also be completely carried out under sterile conditions. The modifications of the above procedure necessary for this, for example with respect to sterilization of the starting materials and the apparatus employed, are known to the person skilled in the art.

### EXAMPLES

The present invention is presented in greater detail below with the aid of non-restrictive preferred examples. Unless indicated otherwise, all quantitative data relate to percentages by weight.

### Example 1

### Lysine Acetylsalicylate

A solution of 9.9 kg of O-acetylsalicylic acid in 120 kg of ethanol is added to a stirring unit container with flow spoiler. At from 20 to 25° C., a solution of 9.0 kg of lysine hydrate and 26.5 kg of water is added within a short period of time and the solutions are mixed such that a temperature of 30° C. is not exceeded. 50 g of seed crystals are added and the already crystallizing mixture is mixed with 120 kg of acetone, with cooling to 0° C. The mixture is allowed to crystallize for 1 to 8 hours with gentle stirring at 0° C. The crystallisate is isolated on a filter or in a centrifuge. The moist product is washed repeatedly with ethanol in a separating apparatus. The moist product is transferred to a dryer and dried therein at a pressure of less than 30 mbar at a temperature of not more than 40° C.

This gave 89 to 94% of the desired product, which had a residual moisture content of from 0.10 to 0.15%.

### Example 2

### D,L-lysine Acetylsalicylate with 10% Glycine

A solution of 9.9 kg O-acetylsalicylic acid in 145 kg of ethanol is added to a stirring unit container with flow spoiler. At from 20 to 25° C., a solution of 9.0 kg of D,L-lysine hydrate and 2.4 kg of glycine in 35 kg of water is added within a short period of time and the solutions are mixed such that a temperature of 30° C. is not exceeded. 50 g of seed crystals are added and the already crystallizing mixture is mixed with 120 kg of acetone, with cooling to 0° C. The mixture is allowed to crystallize for one to eight hours with gentle stirring at 0° C. The crystallisate is isolated on a filter or in a centrifuge. The moist product is washed repeatedly in succession with ethanol and acetone in a separating apparatus. The moist product is transferred to a dryer and dried therein at a pressure of less than 30 mbar at a temperature of not more than 40° C.

This gave 90 to 95% of the desired product, which had a residual moisture content of from 0.10 to 0.15%.

### Example 3

### D,L-lysine Acetylsalicylate with 10% Glycine

A solution of 9.9 kg of O-acetylsalicylic acid in 120 kg of ethanol is added to a stirring unit container with flow spoiler. At from 20 to 25° C., a solution of 9.0 kg of lysine hydrate in 26.5 kg of water is added within a short period of time and the solutions are mixed such that a temperature of 30° C. is not exceeded. 50 g of seed crystals are added and the already crystallizing mixture is mixed with 120 kg of acetone, with cooling to 0° C. The mixture is allowed to crystallize for one to eight hours with gentle stirring at 0° C. In a separate stirring unit container, a suspension of 2.1 kg of glycine in 8 kg of water and 25 kg of ethanol is prepared. This suspension is added to the salicylate suspension. The crystal mixture is isolated on a filter or in a centrifuge. The moist product is repeatedly washed with ethanol in a separating apparatus. The moist product is transferred to a dryer and dried therein at a pressure of less than 30 mbar at a temperature of not more than 40° C.

This gave 89 to 94% of the desired product, which had a residual moisture content of from 0.10 to 0.15%.

The product was used according to Examples 4 to 6 for preparing chewable tablets, tablets and capsules.

Determination of the Particle Size Distribution

The lysine acetylsalicylate from Example 1 and commercially available Aspisol® (marketed by Bayer AG) were investigated in a Malvern 2600 D measuring apparatus from Malvern under the following standard conditions:

The Malvern 2600 measuring apparatus consists of an He/Ne laser, a measuring cuvette having a thermostatted reservoir system, Fourier lenses and multi-element detector. The measured light intensities are converted into a particle size distribution. The alignment of laser and lens are adjusted manually before each measurement and the measuring apparatus is checked by means of a blank measurement. The blank pulses must not exceed a maximum value of 20 per detector element.

The sample to be investigated is shaken by hand for about 15 s; a sample is then taken with a spatula. The amount of sample depends on the permissible obscuration area (0.1-0.3) of the measuring apparatus. The sample taken is gently predispersed in a beaker (by stirring with a glass rod) using a customary dispersing agent such as Baysilon M10® (Bayer AG) and then filled into the reservoir of the measuring apparatus, which is likewise filled with the dispersant. The beaker is rinsed out completely with the dispersant in order to ensure representative sampling.

The measurement is carried out using a set focal length of 300 mm, thermostatting at 20° C. and a permissible obscuration area of 0.1-0.3.

The product is measured after ultrasonication times of 0, 15 and 60 seconds. For this, the ultrasonic finger is situated in the reservoir of the circulating product. The suspension is pumped through the measuring cuvette in a closed circuit. The signals recorded by the detector are analysed and converted into the particle size distribution.

The results thus obtained are shown in FIGS. 1 and 2.

### Example 4

### Chewable Tablets

In this example, the preparation of chewable tablets according to the present invention is described. In addition to improved-flow properties, the saccharides used improve the taste. The taste is masked by combining two sweeteners.

The active compounds and auxiliaries were weighed out, mixed in a Turbula mixer for 10 minutes and, for drying, aspirated with dry air for 12-24 hours. Compaction was carried out in a conventional tablet press. Compaction can be carried out in all conventional tablet presses (for example eccentric and rotating tablet presses).

| | Amount per | Amount per |
|---|---|---|
| Ingredient | tablet [mg] | tablet [mg] |
| Composition from Ex. 3 | 1 000.00 | 1 000.00 |
| Xylitol | 460.00 | — |
| Sorbitol | — | 460.00 |
| Magnesium stearate | 32.00 | 32.00 |
| Aspartame | 4.00 | 4.00 |
| Saccharin | 4.00 | 4.00 |
| Tablet mass [mg] | 1 500.00 | 1 500.00 |

The tablets; can be sealed in vapour-tight composite films (for example PAP-Surlyn composite films) or in aluminium/aluminium blister packs.

### Example 5

### Tablets

In this example, the preparation of tablets according to the present invention is described. In addition to improved flow properties, the saccharides used improve the taste.

The active compounds and auxiliaries were weighed out, mixed in a Turbula mixer for 10 minutes and, for drying, aspirated with dry air for 12-24 hours. Compaction was carried out in a conventional tablet press with external lubrication. Compaction can be carried out in all conventional tablet presses with external lubrication (for example eccentric and rotating tablet presses).

| | Amount | Amount | Amount |
|---|---|---|---|
| | per tablet | per tablet | per tablet |
| Ingredient | [mg] | [mg] | [mg] |
| Composition from | 200.00 | 200.00 | 200.00 |
| Ex. 3 | | | |
| Sorbitol | 40.00 | — | — |
| Xylitol | — | 40.00 | — |
| Mannitol | — | — | 40.00 |
| Tablet mass [mg] | 240.00 | 240.00 | 240.00 |

The tablets can be sealed in vapour-tight composite films (for example PAP-Surlyn composite films) or in aluminium/aluminium blister packs.

Since the tablets comprise only water-soluble active compounds and auxiliaries, the tablets prepared according to the invention can also be used and administered as soluble tablets.

### Example 6

### Capsules

In this example, the preparation of capsules according to the present invention is described. The auxiliaries used serve to improve flow properties. The amount of auxiliaries used depends on the size of the capsule and has to be adjusted for each individual case. The capsules employed were two-piece capsules of different sizes made of hard gelatin and of hydroxypropylmethylcellulose. It is also possible to dry the capsules used prior to filling, thus reducing their content of free water.

Taking into account the size of the capsule, the active compounds and auxiliaries were weighed out, mixed in a Turbula mixer for 10 minutes and, for drying, aspirated with dry air for 12-24 hours. Encapsulation was carried out in a conventional capsule filling machine. It can be carried out in all conventional capsule filling machines (both manual and automatic capsule filling machines).

| | Amount per | Amount per |
|---|---|---|
| Ingredient | capsule [mg] | capsule [mg] |
| Composition from Ex. 3 | 200 mg | 200 mg |
| Mannitol | q.s. | q.s. |
| Capsule material | HPMC capsules | Hard gelatin capsules |

The capsules can be sealed in vapour-tight composite films (for example PAP-Surlyn composite films) or in aluminium/aluminium blister packs.

Determination of the Stability

The stability of the single-dose solid oral pharmaceuticals of Examples 4 to 6 prepared according to the invention was determined after storage at a temperature of 25° C. and a relative atmospheric humidity of 60%. As an indicator for the stability of the preparations, the content of salicylic acid as degradation product of O-acetylsalicylate was determined.

Stability of the Formulations

The stability of the formulations is shown in the diagrams of FIGS. 3 to 5. It can be seen that the preparations according to the invention have a satisfactory shelf-life and that their rate of degradation is comparable to that of the O-acetylsalicylic acid salt with lysine.

FIG. 3 shows the degradation of O-acetylsalicylate to salicylic acid in chewable tablets according to Example 4 when stored at 25° C. and 60%-relative humidity in comparison to the composition of Example 3.

FIG. 4 shows the degradation of O-acetylsalicylate to salicylic acid in tablets according to Example 5 when stored at 25° C. and 60% relative humidity in comparison to the composition of Example 3.

FIG. 5 shows the degradation of O-acetylsalicylate to salicylic acid in capsules, according to Example 6 when stored at 25° C. and 60% relative humidity in comparison to the composition of Example 3.

1. Composition, comprising a salt of O-acetylsalicylic acid with a basic amino acid, which salt has an average particle size above a particle size of 160 μm and a proportion of more than 60% of the particles having a particle size in a range from 100 to 200 μm in a particle size distribution measured using a Malvern 2600D apparatus under standard conditions, characterized in that the composition additionally comprises a flow improver or is granulated.

2. Composition according to claim 1, characterized in that it comprises, as flow improver, one or more saccharides.

3. Composition according to claim 2, characterized in that it is dry-granulated.

4. Composition according to claim 1, characterized in that the salt has an average particle size above a particle size of 170 μm and a proportion of more than 70% of the particles having a particle size in a range from 100 to 200 μm in a particle size distribution measured using a Malvern 2600D apparatus under standard conditions.

5. Composition according to claim 1, characterized in that the basic amino acid is lysine, arginine, histidine, ornithine or diaminobutyric acid.

6. Composition according to claim 1, characterized in that it additionally comprises a proportion of from 5 to 15% by weight of glycine, based on the total amount of O-acetylsalicylate and glycine.

7. Pharmaceutical composition, comprising at least one composition according to claim 1.

8. Pharmaceutical composition according to claim 7, characterized in that it is provided as a single-dose solid oral administration form.

9. A pharmaceutical composition as claimed in claim 7, characterized in that it only comprises water-soluble auxiliaries.

10. Pharmaceutical composition according to claim 7, characterized in that it is completely soluble in water.

11. Pharmaceutical according to claim 7, characterized in that it comprises one or more further pharmaceutically active compounds.

12. A method of treating disorders of a rheumatic type, arthritis, neuralgia, myalgia or migraine, comprising administering to a patient in need thereof an effective amount of a composition of claim 1.

13. A method of treating treating ischaemic heart diseases, stroke, angina pectoris, myocardial infarction, bypass operations, PTCA or stent implants, comprising administering to a patient in need thereof an effective amount of a composition of claim 1.

14. A method for stimulating the immune system of HIV patients, for tumour prophylaxis, for slowing down the cognitive deterioration associated with dementia, for inhibiting the formation of gallstones or for treating diabetic disorders, comprising administering to a patient in need thereof an effective amount of a composition of claim 1.

15. The pharmaceutical composition of claim 8, wherein the composition is a tablet, a chewable tablet, a soluble tablet, an enteric-coated tablet, a capsule or a colon-targeted formulation.

16. The pharmaceutical of claim 11, wherein the pharmaceutically active compound is selected from ADP receptor antagonists, GPIIb/IIIa receptor antagonists, phosphodiesterase inhibitors, thrombin receptor antagonists, factor Xa inhibitors, HMG-CoA receptor antagonists and calcium antagonists.

17. The composition of claim 2, wherein the flow improver is selected from the group consisting of mannitol, sorbitol, xylitol, and lactose, and a mixture thereof.

18. The composition of claim 3, wherein the composition is roller-compacted.

19. The pharmaceutical composition of claim 9, wherein the water-soluble auxiliary is a flow improver selected from the group consisting of mannitol, sorbitol, xylitol, and lactose, and a mixture thereof.
